(19) United States
(12) Patent Application Publication
(10) Pub. No.: US 2026/0325191 A1
(43) Pub. Date: Oct. 22, 2026

(54) SURGICAL SCREW ALIGNER AND METHODS OF USE THEREOF

(71) Applicant: Board of Supervisors of Louisiana State University and Agricultural and Mechanical College, Baton Rouge, LA (US)

(72) Inventors: Peter C. Krause, New Orleans, LA (US); Charles E. Taylor, New Orleans, LA (US)

(21) Appl. No.: 19/112,097

(22) PCT Filed: Sep. 18, 2023
(86) PCT No.: PCT/US2023/074447

§ 371 Date: Mar. 14, 2025

Related U.S. Application Data
(60) Provisional application No. 63/407,479, filed on Sep. 16, 2022.

(51) Int. Cl. A61B 17/90; A61B 17/72; A61B 17/84

(52) U.S. Cl. CPC A61B 17/90; A61B 17/72; A61B 17/848

(57) ABSTRACT

A surgical alignment device is described herein comprising an alignment head, wherein the alignment head comprises a plurality of tool access holes, a k-wire hole array comprising a first plurality of k-wire holes and a second plurality of k-wire holes, wherein the first plurality of k-wire holes extends radially from a first surface of the alignment head to a second surface of the alignment head, wherein the second plurality of k-wire holes are parallel to the plurality of tool access holes.

This application is an International Application which claims priority to U.S. Application No. 63/407,479, filed Sep. 16, 2022.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to a surgical screw aligner and methods of using the same.

### BACKGROUND OF THE INVENTION

Blocking screws are used at the proximal and/or distal end of a long bone to provide alignment of a nail prosthesis and delivery structural reinforcement. Current practice is to free-hand the placement of the blocking screws, which consumes both time and intraoperative x-ray. Both effects are undesirable for products used in trauma cases.

### SUMMARY OF THE INVENTION

In embodiments, a surgical alignment device is described herein comprising an alignment head, wherein the alignment head comprises at least two tool access holes, a k-wire hole array comprising holes parallel to the tool access holes, and a handle attachment pocket perpendicular to the tool access holes.

In embodiments, the k-wire hole array comprises an arrangement of holes in an arc pattern surrounding the tool access holes.

In embodiments, the arc pattern comprises radially distributed groupings of holes about the alignment head.

In embodiments, the radially distributed groupings comprise at least one first grouping of holes.

In embodiments, the radially distributed groupings comprise at least one second grouping of holes.

In embodiments, locations of holes in the at least first grouping are laterally transposed from locations of holes in the at least second grouping.

In embodiments, locations of holes in the at least first grouping are longitudinally transposed from locations of holes in the at least second grouping.

In embodiments, the k-wire hole array comprises an arrangement of k-wire holes longitudinally aligned to follow a tibial nail axis.

In embodiments, the handle attachment pocket further comprises retainment pen holes.

In embodiments, the handle is attachable to the alignment head with the placement of a retainment pen in the retainment pen holes.

In embodiments, a surgical alignment device is described herein comprising an alignment head, wherein the alignment head comprises a plurality of tool access holes, a k-wire hole array comprising a first plurality of k-wire holes and a second plurality of k-wire holes, wherein the first plurality of k-wire holes extends radially from a first surface of the alignment head to a second surface of the alignment head, wherein the second plurality of k-wire holes are parallel to the plurality of tool access holes.

In embodiments, the plurality of tool access holes comprises a first and second pair of tool access holes.

In embodiments, the first pair of tool access holes comprises a first proximal tool access hole and a first distal tool access hole.

In embodiments, the second pair of tool access holes comprises a second proximal tool access hole and a second distal tool access hole.

In embodiments, centers of the first and second proximal tool access holes laterally oppose each other at a first distance.

In embodiments, centers of the first and second distal tool access holes laterally oppose each other at a second distance, wherein the first distance is greater than the second distance.

In embodiments, the first pair of tool access holes are joined in fluid communication through a shared open pathway extending along their respective lengths.

In embodiments, the second pair of tool access holes are joined in fluid communication through a shared open pathway extending along their respective lengths.

In embodiments, the first and second pair of tool access holes comprise semi-circular access extensions in respective proximal and distal directions.

In embodiments, the first plurality of k-wire holes are laterally distributed along opposing sides of the alignment head.

In embodiments, the second plurality of k-wire holes are longitudinally distributed between the first and second pair of tool access holes.

In embodiments, the first plurality of k-wire holes are radially separated from each other at five degrees.

In embodiments, the first plurality of k-wire holes are radially separated end to end at thirty degrees.

In embodiments, the second plurality of k-wire holes occupy a second plane, wherein the first plurality of k-wire holes occupy two respective parallel planes, wherein the first plane is orthogonal to the two respective parallel planes.

In embodiments, a handle attachment laterally extends from the alignment head, wherein the handle attachment is configured to receive a handle in a press fit coupling.

In embodiments, a method is described herein comprising configuring an alignment device for placement of at least one blocking screw, wherein the alignment head comprises a plurality of tool access holes, a k-wire hole array comprising a first plurality of k-wire holes and a second plurality of k-wire holes, wherein the first plurality of k-wire holes extends radially from a first surface of the alignment head to a second surface of the alignment head, wherein the second plurality of k-wire holes are parallel to the plurality of tool access holes.

In embodiments, the plurality of tool access holes comprises a first and second pair of tool access holes.

In embodiments, the first pair of tool access holes comprises a first proximal tool access hole and a first distal tool access hole.

In embodiments, the second pair of tool access holes comprises a second proximal tool access hole and a second distal tool access hole.

In embodiments, centers of the first and second proximal tool access holes laterally oppose each other at a first distance.

In embodiments, centers of the first and second distal tool access holes laterally oppose each other at a second distance, wherein the first distance is greater than the second distance.

In embodiments, the first pair of tool access holes are joined in fluid communication through a shared open pathway extending along their respective lengths.

In embodiments, the second pair of tool access holes are joined in fluid communication through a shared open pathway extending along their respective lengths.

In embodiments, the first and second pair of tool access holes comprise semi-circular access extensions in respective proximal and distal directions.

In embodiments, the first plurality of k-wire holes are laterally distributed along opposing sides of the alignment head.

In embodiments, the second plurality of k-wire holes are longitudinally distributed between the first and second pair of tool access holes.

In embodiments, the first plurality of k-wire holes are radially separated from each other at five degrees.

In embodiments, the first plurality of k-wire holes are radially separated end to end at thirty degrees.

In embodiments, the second plurality of k-wire holes occupy a second plane, wherein the first plurality of k-wire holes occupy two respective parallel planes, wherein the second plane is orthogonal to the two respective parallel planes.

In embodiments, a handle attachment laterally extends from the alignment head, wherein the handle attachment is configured to receive a handle in a press fit coupling.

In embodiments, a method is described herein of guiding blocking screws into a bone of a subject comprising placing the surgical alignment device of any one of claims 1-40 on a subject to align with a bone to which at least one blocking screw is to be administered, anchoring the device on the subject by placement of at least one k-wire, and inserting a tool through the surgical alignment device.

In embodiments, the blocking screws are administered prior to a reduction of a bone fracture.

In embodiments, the blocking screws are administered prior to a nail insertion.

In embodiments, the bone comprises a tibia.

In embodiments, the bone comprises a femur.

Other objects and advantages of this invention will become readily apparent from the ensuing description.

### BRIEF DESCRIPTION OF THE FIGURES

FIG. 1 shows a schematic of Tibial Nail System showing locking screws at the proximal and distal point (near foot), under an embodiment.

FIG. 2 shows a schematic of Distal femoral nail with locking screws at the distal end (bottom, closer to knee), under an embodiment.

FIG. 3 shows a schematic of Humeral nail system with only locking screws at each end.

FIG. 4 shows a schematic of Smith & Nephew blocking screw system, under an embodiment.

FIG. 5 shows a perspective view of an LSUHSC Blocking Screw Aligner (R2 design), under an embodiment.

FIG. 6A shows a top aspect CT scan of a blocking screw alignment device in use for positioning k-wires and blocking screws, under an embodiment.

FIG. 6B shows a bottom aspect CT scan of a blocking screw alignment device in use for positioning k-wires and blocking screws, under an embodiment.

FIG. 6C shows a bottom aspect CT scan of a blocking screw alignment device in use for positioning k-wires and blocking screws, under an embodiment.

FIG. 6D shows a top aspect CT scan of a blocking screw alignment device in use for positioning k-wires and blocking screws, under an embodiment.

FIG. 6E shows a side aspect CT scan of a blocking screw alignment device in use for positioning k-wires and blocking screws, under an embodiment.

FIG. 6F shows a top aspect CT scan of a blocking screw alignment device in use for positioning k-wires and blocking screws, under an embodiment.

FIG. 6G shows a top aspect CT scan of a blocking screw alignment device in use for positioning k-wires and blocking screws, under an embodiment.

FIG. 6H shows a side aspect CT scan of a blocking screw alignment device in use for positioning k-wires and blocking screws, under an embodiment.

FIG. 7A shows another shows a perspective view of an LSUHSC Blocking Screw Aligner (R2 design), under an embodiment.

FIG. 7B shows a top view of an LSUHSC Blocking Screw Aligner (R2 design), under an embodiment.

FIG. 8 shows a photograph of Initial placement of R2 design on cadaver, under an embodiment.

FIG. 9 shows a photograph of Initial k-wire anchoring on R2, under an embodiment.

FIG. 10 shows a photograph of Alternate view of initial k-wire placement on R2, under an embodiment.

FIG. 11 shows a photograph of K-wires and trocar set, under an embodiment.

FIG. 12A shows a schematic of a Krause_DrillGuide_R2, under an embodiment.

FIG. 12B shows a perspective view of Krause_DrillGuide_R2, under an embodiment.

FIG. 12C shows a perspective view of Krause_DrillGuide_R2, under an embodiment.

FIG. 12D shows a perspective view of Krause_DrillGuide_R2, under an embodiment.

FIG. 13A shows a schematic of a Krause_DrillGuide_R1A, under an embodiment.

FIG. 13B shows a perspective view of a Krause_DrillGuide_R1A, under an embodiment.

FIG. 13C shows a perspective view of a Krause_DrillGuide_R1A, under an embodiment.

FIG. 13D shows a perspective view of Krause_DrillGuide_R1A, under an embodiment.

FIG. 14A shows a schematic of a Krause_DrillGuide_R1B, under an embodiment.

FIG. 14B shows a perspective view of a Krause_DrillGuide_R1B, under an embodiment.

FIG. 14C shows a perspective view of a Krause_DrillGuide_R1B, under an embodiment.

FIG. 14D shows a perspective view of Krause_DrillGuide_R1B, under an embodiment.

FIG. 15A shows a perspective view of a Krause_DrillGuide_R8, under an embodiment.

FIG. 15B shows a perspective view of a Krause_DrillGuide_R8, under an embodiment.

FIG. 16A shows a perspective view of a Krause_DrillGuide_R10, under an embodiment.

FIG. 16B shows a perspective view of a Krause_DrillGuide_R10, under an embodiment.

FIG. 16C shows a schematic top view of a Krause_DrillGuide_R10, under an embodiment.

FIG. 16D shows a schematic partial top of a Krause_DrillGuide_R10, under an embodiment.

FIG. 16E shows a schematic cross sectional side view of a Krause_DrillGuide_R10, under an embodiment.

### DETAILED DESCRIPTION OF THE INVENTION

### Abbreviations and Definitions

Detailed descriptions of one or more preferred embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. The spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

As used herein, the term “articulate” can refer to moving or movable and comprises all degrees of translational displacements and/or rotations. For example, the articulation can be axial, longitudinal, forward, backward, orthogonal, lateral, transverse, rotational, pivotable, sloping incline or decline, swinging, torsional, revolving, and other forms of translation and/or rotation in an x, y, and/or z coordinate system (collectively, “articulation”, “articulate”, “articulatable”, and variants thereof).

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Orthopedic Blocking Screw Aligner

Described herein is a surgical-grade 3D-printed aligner to improve the success and simplify the surgical treatment of distal/proximal femur and tibia fractures. In embodiments, aspects of the invention can be used in surgery to guide the placement blocking screws within the bone of the tibia or femur. These blocking screws align the intramedullary nails to stabilize the fractures.

Blocking screws are used at the distal end of a long bone to provide alignment of a nail prosthesis and deliver structural reinforcement. Current practice is to free-hand the placement of the blocking screws, which consumes both time and intraoperative x-ray. Both effects are undesirable for products used in trauma cases. Intraoperative x-ray exposure has become a concern amongst surgeons as cancer rates are becoming more visible in this profession and health systems are developing programmatic responses to radiation exposure in personnel. The only blocking screw alignment device is designed to deliver the screws after the nail is implanted; this does not provide the insertion guidance nor structural reinforcement during the placement. There is a need to deliver a solution that can: (1) align two blocking screws to the distal section of a long bone, (2) place those elements prior to the nail insertion, and (3) reduce the time required for accurate placement.

Without wishing to be bound by theory, we can incorporate a blocking screw alignment technology that addresses the need of the surgeons and delivers clinical precision in this method to a wider range of surgical proficiencies.

Non-limiting benefits comprise clinical precision performance from a wider range of proficiencies, reduced radiation exposure in their personnel, and better implant performance with 2 blocking screws lowers the probability of complications.

Non-limiting benefits to surgeons utilizing this technology are: repeatable clinical precision earlier in their career, reduced radiation exposure via an equipment package that accelerates placement, and reduced liability in their implant performance with better patient outcome scores.

Benefits of the disclosure comprise low amount of setup time and reduction in complexity (opposed to Smith & Nephew system), incorporation of parts already in surgical tray (e.g., k-wires), and use of x-ray for alignment (trusted, ubiquitous). Aspects of the invention can meet these needs and requested attributes.

Aspects of the invention comprise a blocking screw alignment device that impacts both the tibial and femoral intermedullary nail. Embodiments can also comprise trauma fixation devices. Embodiments of the blocking screw alignment technology described herein have wide societal need in populations requiring cost-effective solutions for precision medicine.

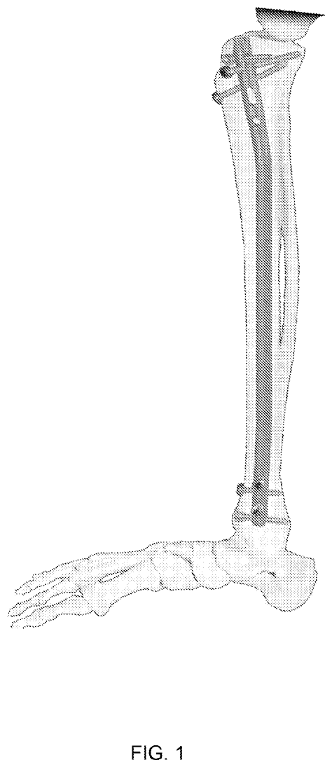

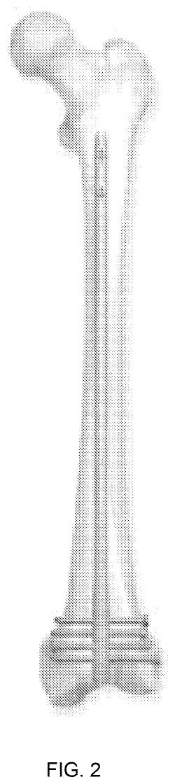

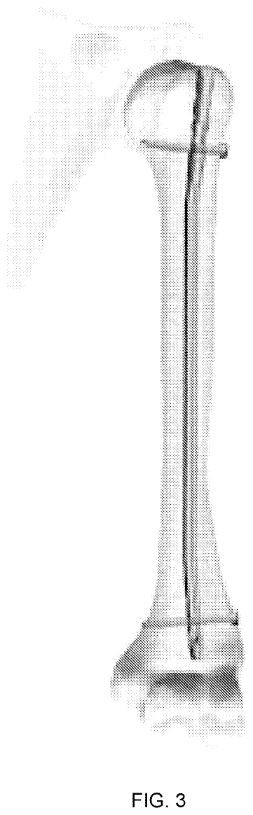

In embodiments, the blocking screw placement for tibial intermedullary nails and design for the distal section can be addressed. Tibial fractures constitute about 2% of all adult fractures in the USA. Roughly 492,000 tibial fractures occur per year resulting in about 500,000 hospital days. Distal tibia fractures represent about 20% of all tibia fractures and proximal tibial fractures are about 50% of all tibial fractures with many receiving nail implants (FIG. 1). These fractures can benefit from dual blocking screw placement. The USA femoral market shows about 250,000 proximal femur fractures occur per year and roughly 30,000 distal femur fractures occur per year. The latter can benefit from blocking screw placement strategies (FIG. 2). The same rationale can apply to humeral nails; development of the concept can encompass this implant system (FIG. 3). Notably, these implant zones have impact on mobility and comorbidity acceleration in the patient if the outcomes are not positive. Thus, even reducing the probability of negative outcomes warrants use of at least 2 additional blocking screws given the marginal cost of this additional hardware and the high cost of hospitalization due to complications.

### Non-limiting Competitive Advantages

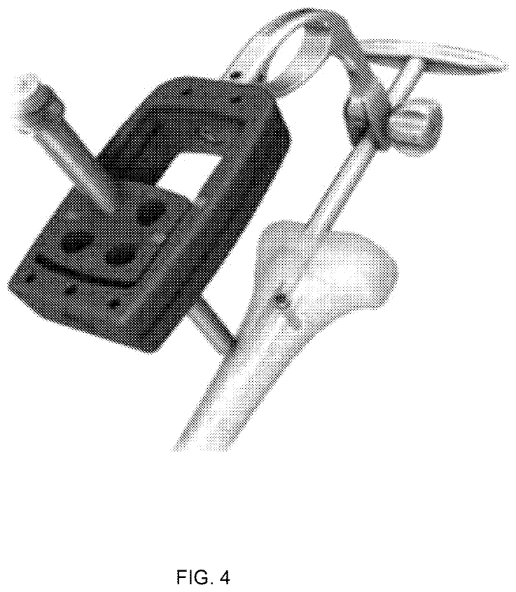

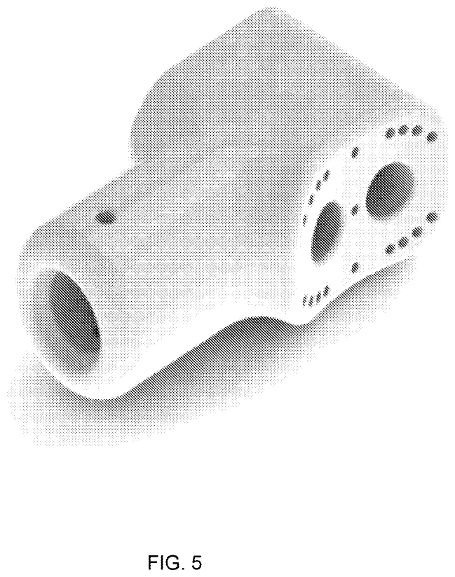

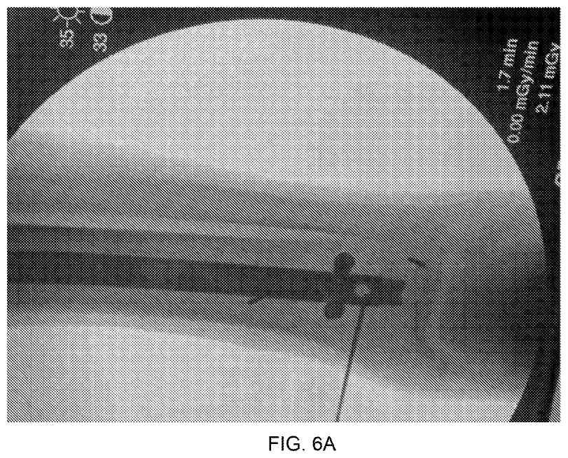

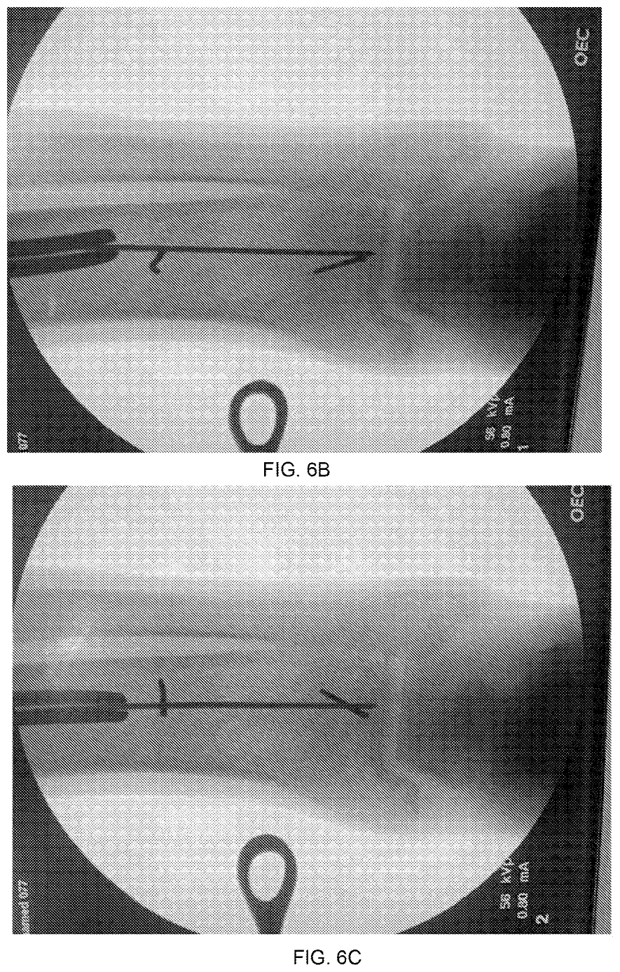

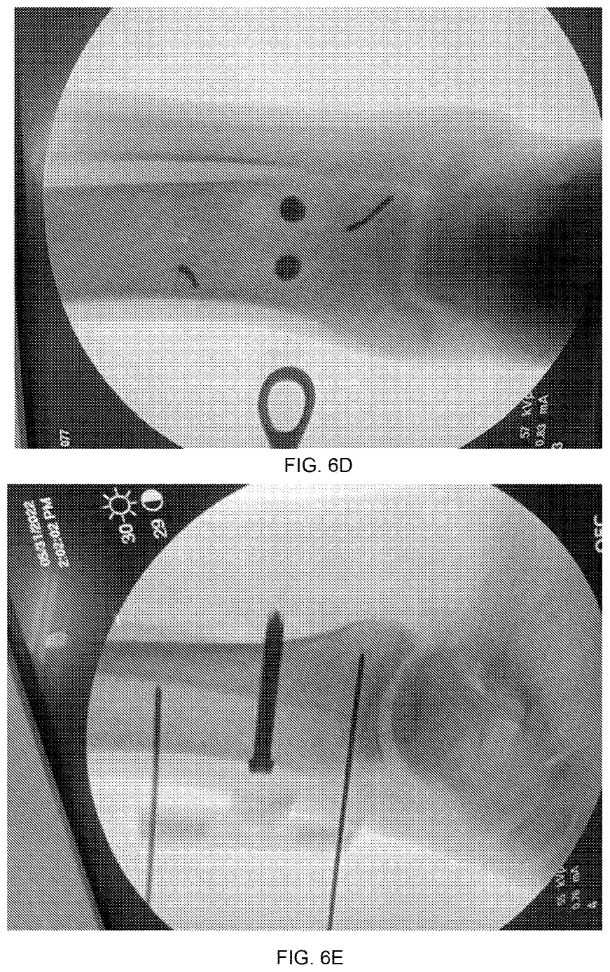

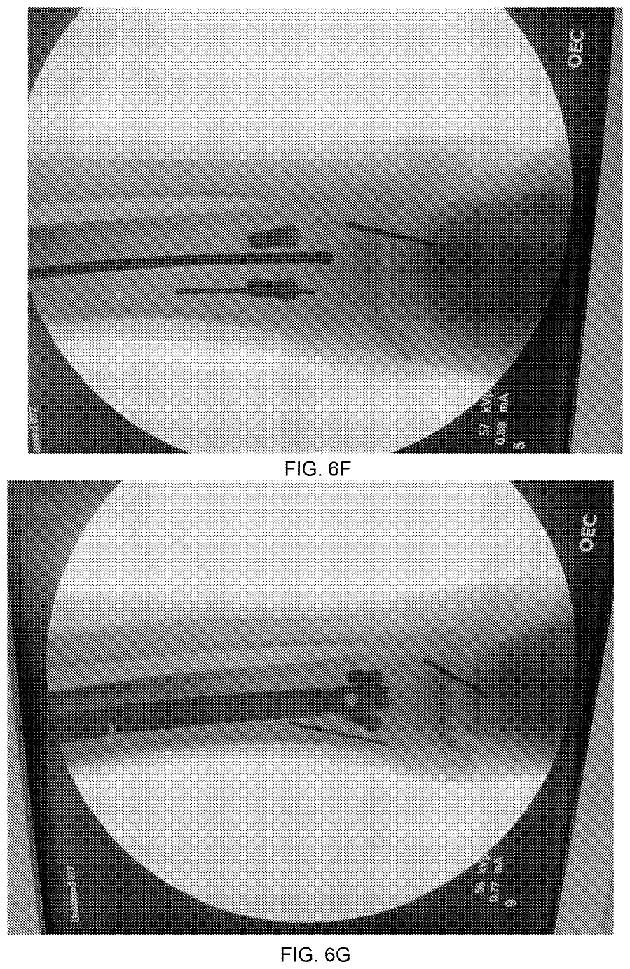

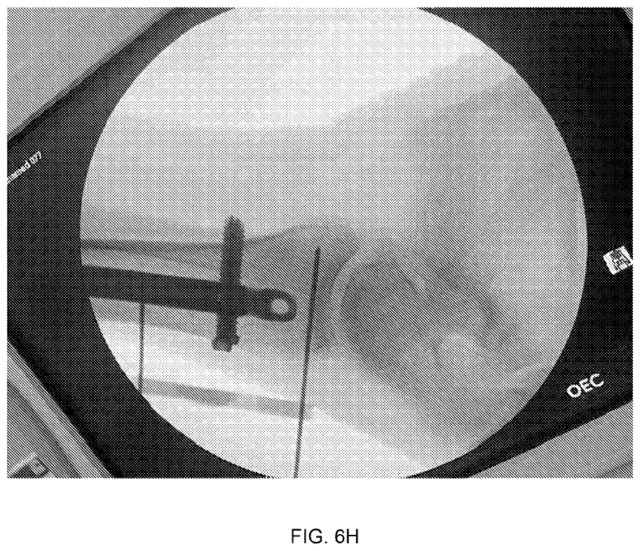

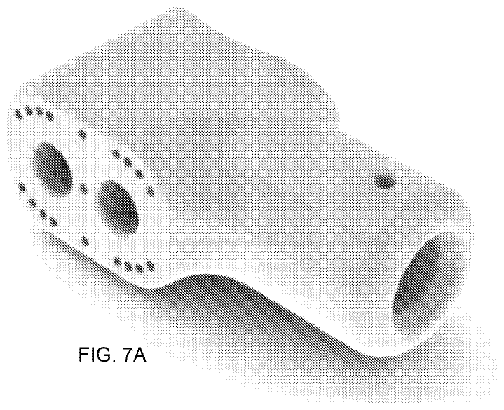

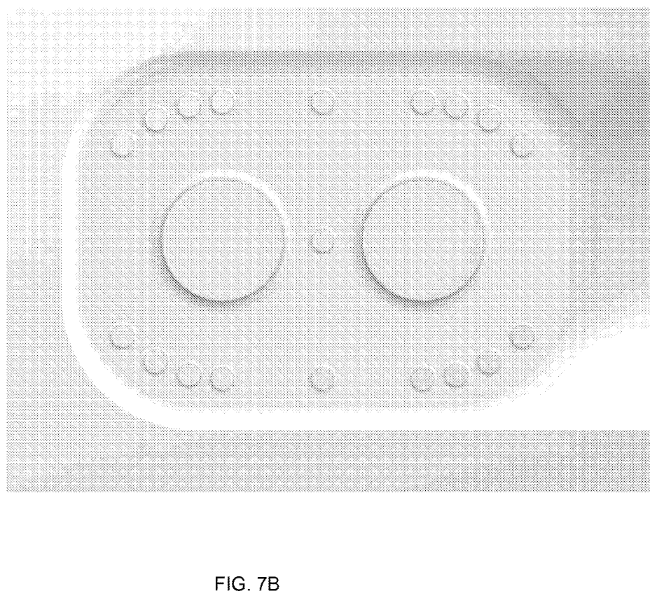

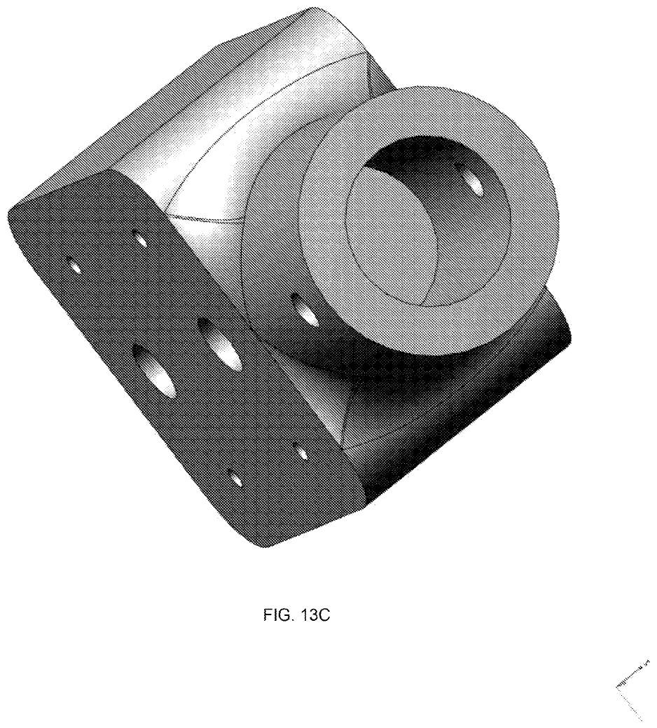

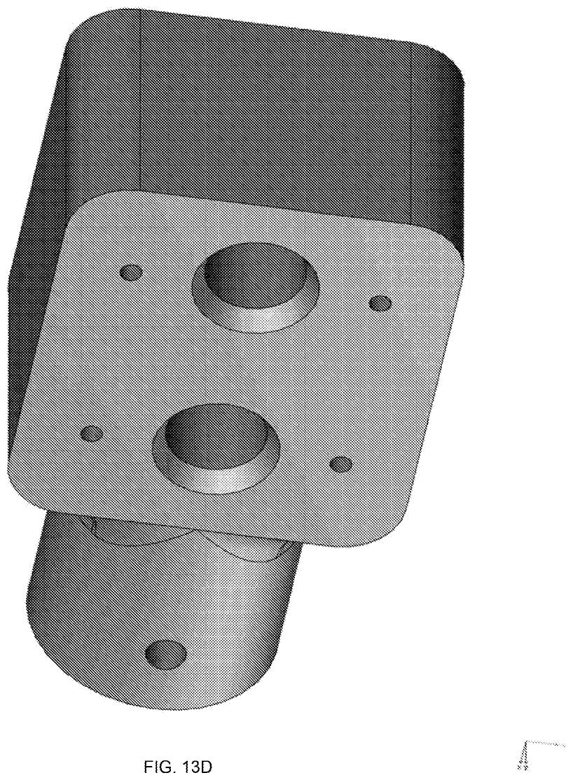

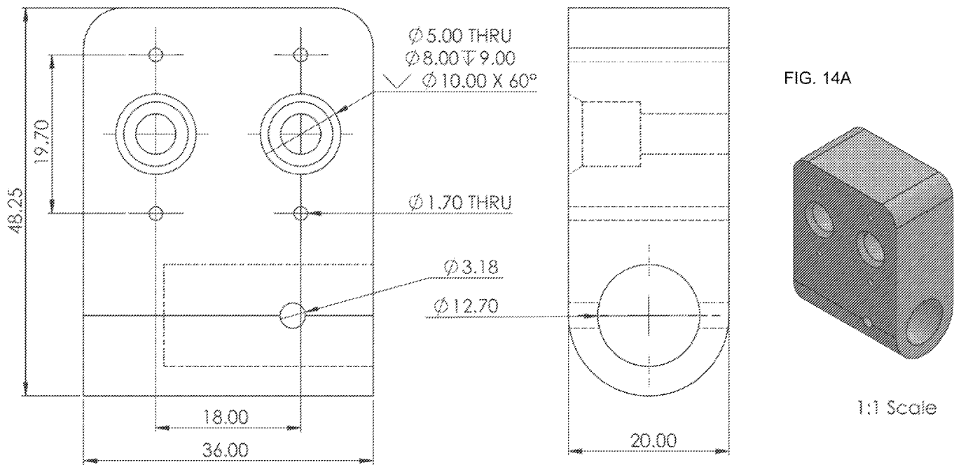

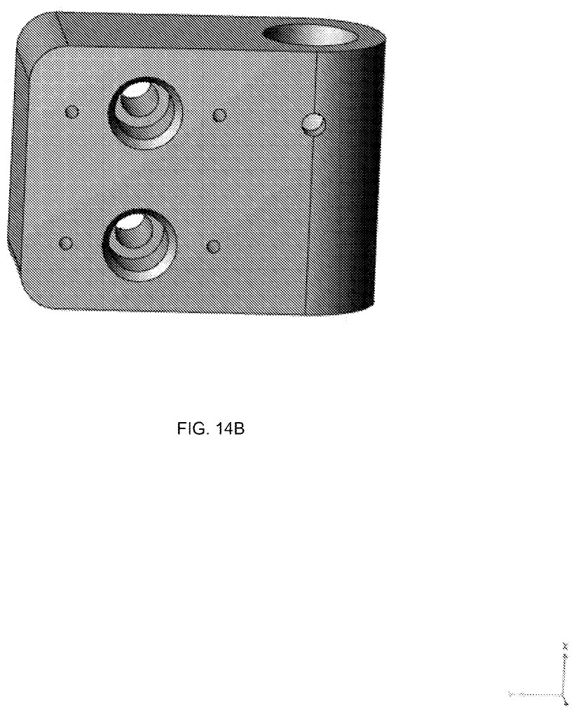

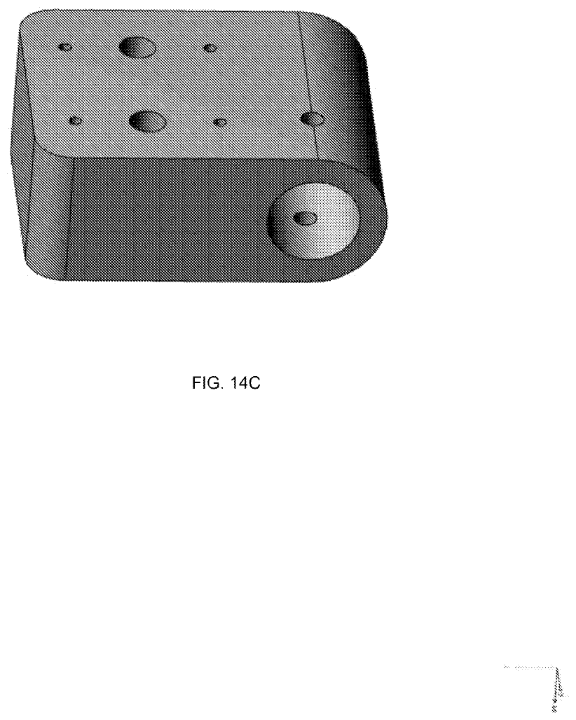

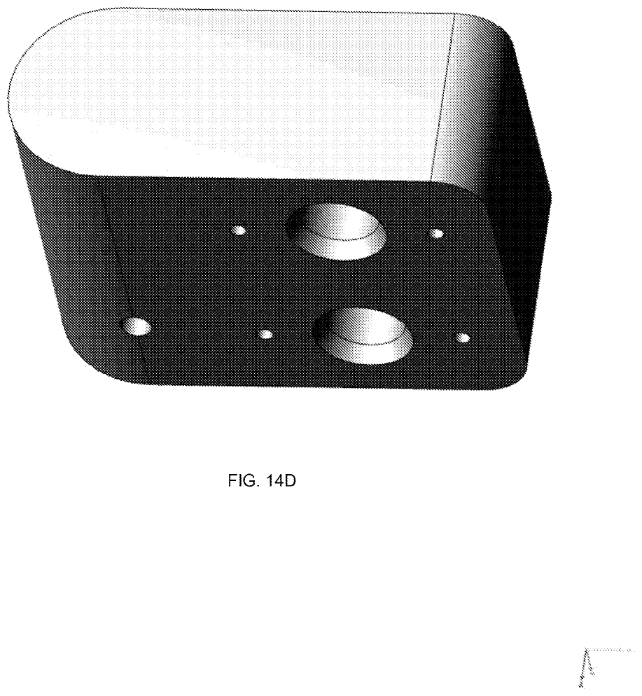

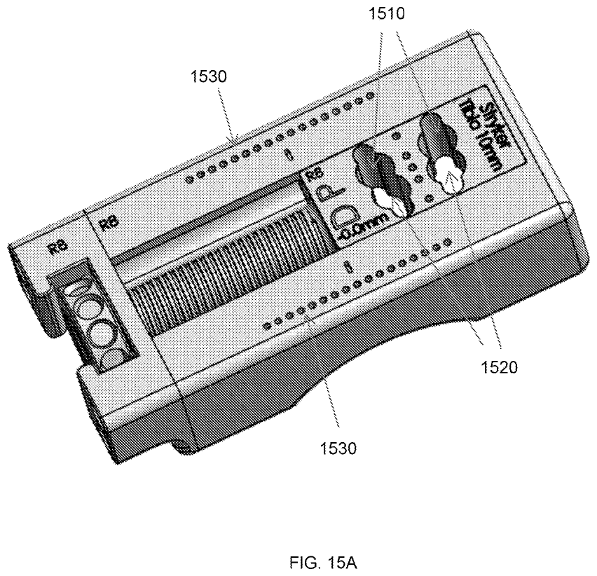

The device design described herein does not exist on the market (FIGS. 5-33). A device is offered by Smith & Nephew; attachment to their targeting jig for the placement of blocking screws after the nail has been inserted (FIG. 4). Their device does not assist in placing blocking screws before the fracture is reduced and the nail is inserted, which is a function for the disclosure described herein. The design described herein is directed (but not limited) to, both (A) strategy of fracture reduction (blocking screws first) and (B) biomechanical reinforcement via at least dual blocking screw placement.

Non-limiting, distinct traits that are a competitive advantage of this device to set it apart comprise: implements standard OR supplies, cost efficiency, radiographic alignment markings, and fast deployment in surgery. The utilization of k-wires in the positioning strategy for this device utilizes this standard equipment to debase the use of expensive rigging systems that is the strategy currently pursued by vendors. This forms the basis of the cost efficiency in this design; small form factor, no need for large articulating systems, and conformal to just-in-time manufacturing concepts to reduce stock need. The fast deployment of this device, since it does not rely on setting large rigging systems, makes it competitive for the trauma market and stakeholders in this arena (DoD, health systems with trauma centers). The confluence of these competitive advantages increases the available target audience; the strategy shift in the use of blocking screws is preferable and the low cost of implementation encompasses traditional stakeholders along with emerging customers (international markets, tight margin health systems).

The blocking screw alignment system described herein can be used in cadaver models.

Without wishing to be bound by theory, the disclosure can provide for devices ready for human validation based on a process of refinement and validation. The refinement process will utilize a V&V40 approach to design that is conformant with industry practices and managed with the DigFabCtr ISO-13485 conformant processes. (The American Society of Mechanical Engineers V&V 40 standard is an FDA-recognized standard that provides a risk-based framework for establishing the credibility requirements of a computational model. ISO-13485 describes the medical industry's optimal medical device standards). Utilizing printing systems in place, the refinement of the design may be verified in biomechanical printed models using tissue mimicking resins and reserve cadaver work for validation.

The DigFabCtr J750 DAP printing system can print multi-material biomechanical models to assess device function against an array of patient presentations and is conducive to performance analysis in vitro with a technology familiar to many device vendor R&D units. Additionally, the same system can produce the FDA-conformant embodiments of this device with its surgical-grade resins. Without wishing to be bound by theory, we can have the surgery-ready prototypes with underlying validation data supporting IRB approval and FDA IDE filings.

The alignment system was designed to have interchangeable alignment heads that share a common mounting to the shaft and handle subsystem; only head pieces need to be printed for updated designs.

### Non-Limiting Research and Development

We can utilize a V&V40 approach conformant to an ISO-13485 documentation process to achieve a validation of the device in a cadaveric model using ISO-10993 compliant resins; data and embodiment ready for a clinical trial. In embodiments, the disclosure can have 3 phases in the project: (A) design data collection, (B) in vitro model, and (C) cadaveric model. A Kaizen continuous improvement process will be employed to guide the feedback (B, C) and properly embody those changes (A).

Stage (A): Design data collection can involve the aggregation of measurements taken experimentally or clinically to drive adjustments in the design of the blocking screw alignment system. Without wishing to be bound by theory, a k-wire alignment pattern and accommodation of k-wire drivers that can be used in clinics can be developed. Validation of the part systems from various vendors to ascertain any geometries that are inconsistent with the design premise of the blocking screw alignment design described herein. Subsequent iterations of this phase will be to validate alignment measurements in the biomechanical cadaveric models of the delivered hardware.

Stage (B): in vitro model validation can involve the use of 3D printed biomechanical models to validate the performance of the design. The DigFabCtr J750 Digital Anatomy Printer can print multimaterial models with resins that mimic human tissues; it can print both soft tissue as well as types of bone (trabecular, cortical). Thus, a cross-sectional model can be delivered that incorporates analogs of all the tissues the device can interact with. Non-limiting benefits of this method are the cost, ability to perform the work outside a sterile setting, and capacity to embody a wide range of morphologies (bone presentations, soft tissue thicknesses) that is impossible to source representative cadavers for at this price point.

Stage (C): cadaveric models will quantify the performance of the device in a system that is as close to the clinical in situ conditions. This stage can characterize the precision of the alignment using the blocking screw jig against randomly assigned cadavers. This test bed will allow the concept to be evaluated against factors of the tibial nail fracture reduction procedure in a high-fidelity model that approximates the end operational environment; cadaver lab stations are equipped with OR-grade imaging and tools.

Other methods such as purchased biomechanical models, animal models, and in silico tools can be utilized. Animal models require divergence in the geometry that is based in human anatomy.

Non-Limiting Formative Design Improvements: Iterative work developing the sizing tables for the device that account for the different vendor part geometries, evaluation against nominal patient anatomy presentations, and assessment in cadaver lab. Outcomes: (i) design allows for use of 2 different vendor tibial nail products in 2 sizes from each vendor (>75% market applicability), (ii) completed Design for Quality (DFQ) processes (House-of-Quality, Kaizen loops), (iii) 3-4 biomechanical model morphologies designed, (iv) at least 12 I-Corp type interviews completed, (v) establish performance expectations for validation.

Design modeled In Vitro: Iterative testing of design concepts with 3D printed biomechanical models from the DigFabCtr J750 Digital Anatomy Printer. Exemplary Outcomes: (i) metrology of blocking screw placement in distal tibia analogs and (ii) feedback from surgeons on the haptics of the device on in vitro models.

Design Modeled in Cadaver: Capstone assessment of device in model system using a full tibial fracture reduction workflow with contact of blocking screws with tibial nail. Without wishing to be bound by theory, exemplary performance: distance of 0.00 to −0.05 mm of medial-lateral nail width and parallelism of <3 degrees.

Without wishing to be bound by theory, we can produce the CAD models, design the biomechanical models, design history file documentation (GD&T, design rationale), print the models, post-process the models, and assist the PI with metrology practices during testing. The PI will need surgical metrology instruments for the cadaver validation. High resolution CT scans can be obtained of the cadaver validation specimens.

### Example 2

### Drill Guide for Tibial Blocking Screws

### Krause_Drillguide_R2

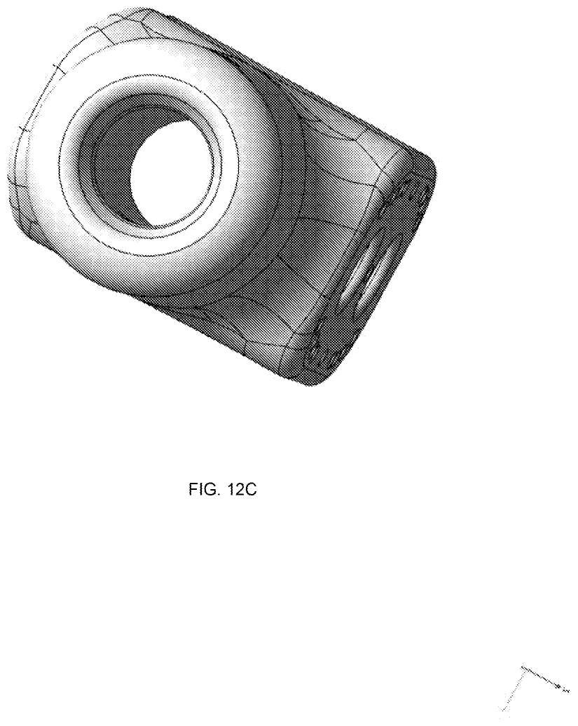

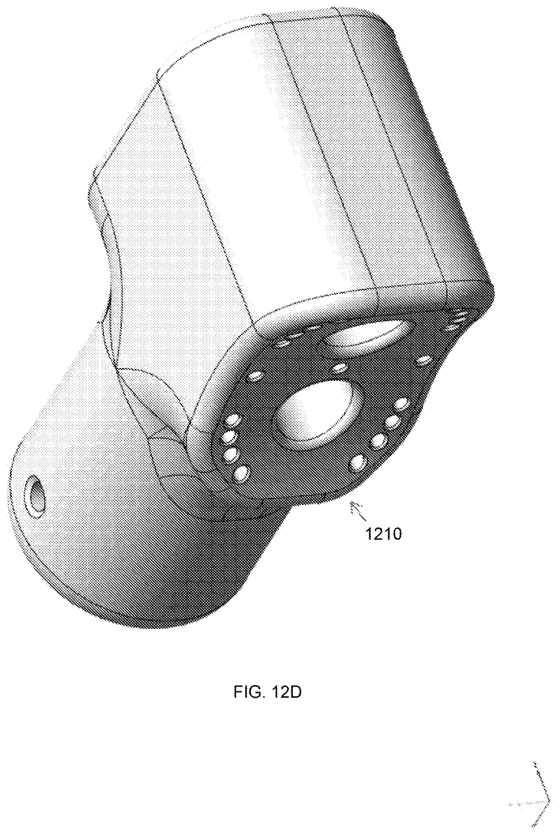

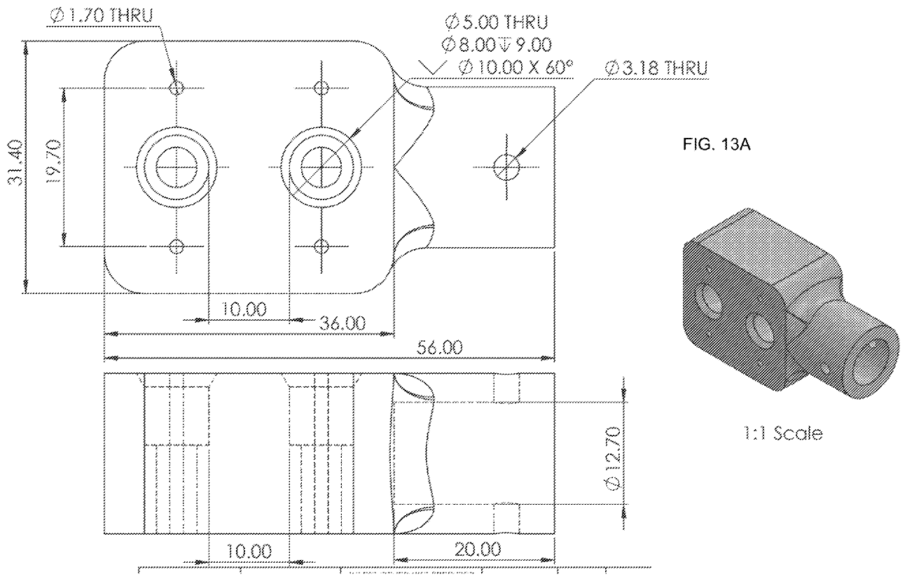

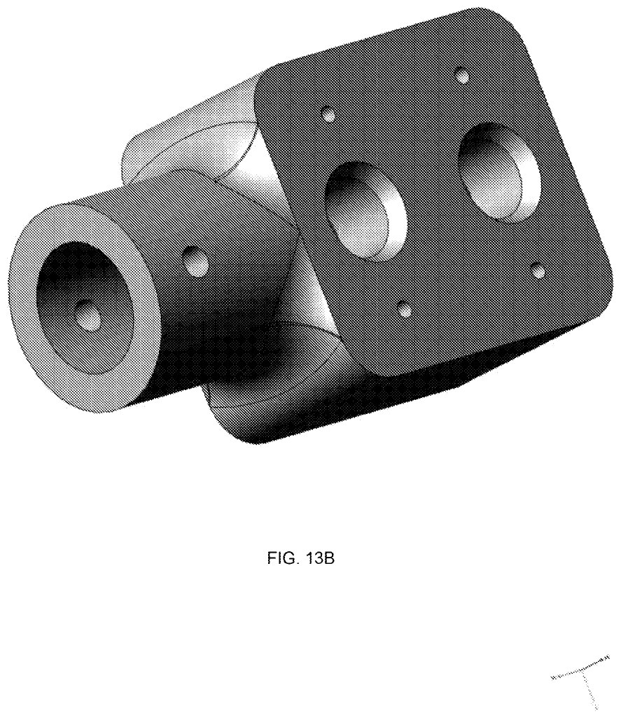

FIG. 12A shows the design of the Krause_Drillguide_R2 (referred to herein as the “R2 design” or the “R2 device”). The design comprises a k-wire hole array allowing for various tibial widths in an arc pattern around tool access holes 1212. The arc pattern features radially distributed groupings 1202, 1204, 1206, 1208 of k-wire holes about a curve skin contact surface 1210. Grouping 1202 and 1204 and grouping 1206 and 1208 each comprise respective series of k-wire hole placements laterally displaced (from outermost to innermost corresponding pairings) at 30.10 mm, 25.10 mm, 20.10 mm, and 15.10 mm. The R2 device also introduces centered k-wire holes 1220 along curve skin surface 1210 that follow a tibial nail axis. Note that the curve skin contact surface 1210 allows better placement of the k-wires and blocking screws.

The tool access holes 1212 accommodate 5 mm diameter screws with a 10.1 mm clearance between screws. The R2 design features 1.70 mm diameter k-wire holes ensuring slip clearance for corresponding k-wires.

The R2 design is configured to receive a handle for manipulation and placement of the blocking screw alignment device. The handle utilizes a circular pocket seat with retainment pin centered tool access holes. The handle comprises an acetal Delrin rod (½″ diameter) cut to length secured with a clevis pin. Adjustment to design of tool access hole is to allow for 9.02 mm OD skin protection sleeve and removal of drill bushings since guide does not interact with rotating tool surface.

### Material: VeroWhite and VeroBlackPlus (Non-Biocompatible)

### Experimental Data and Reduction to Practice of R2

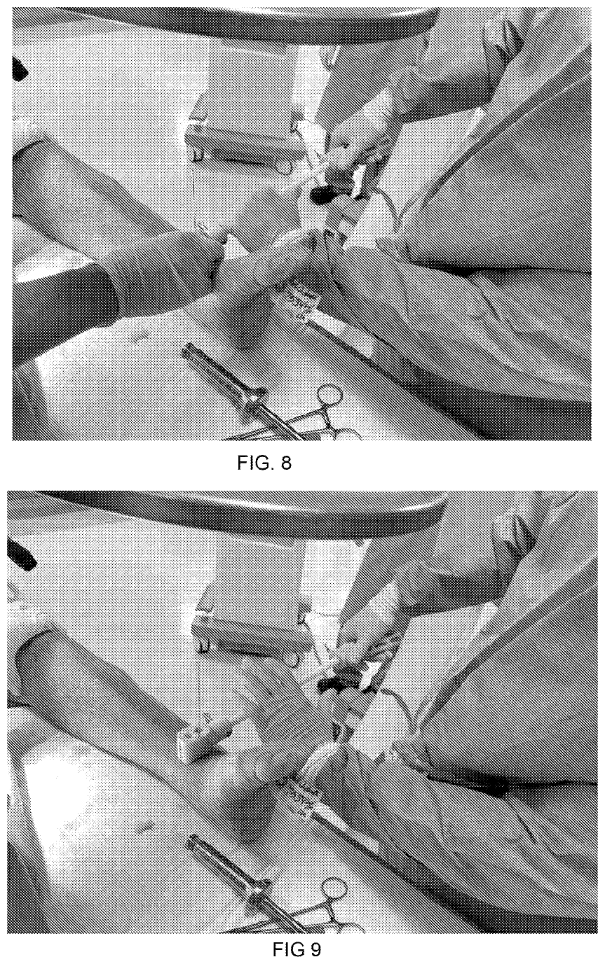

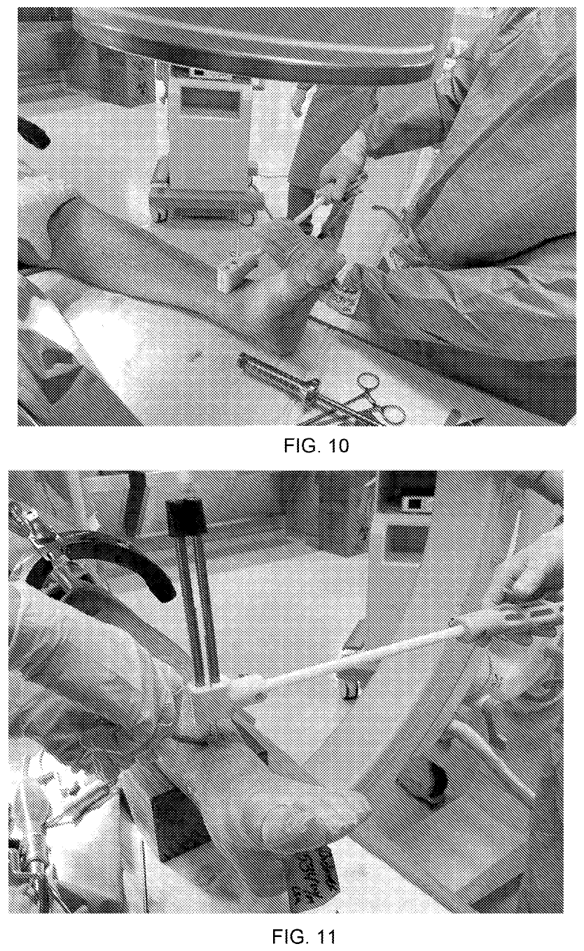

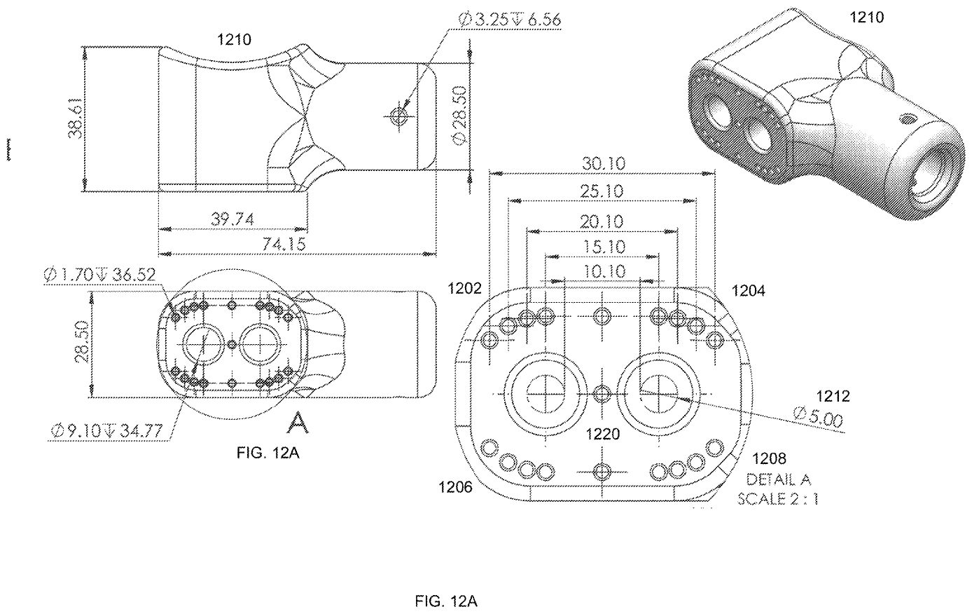

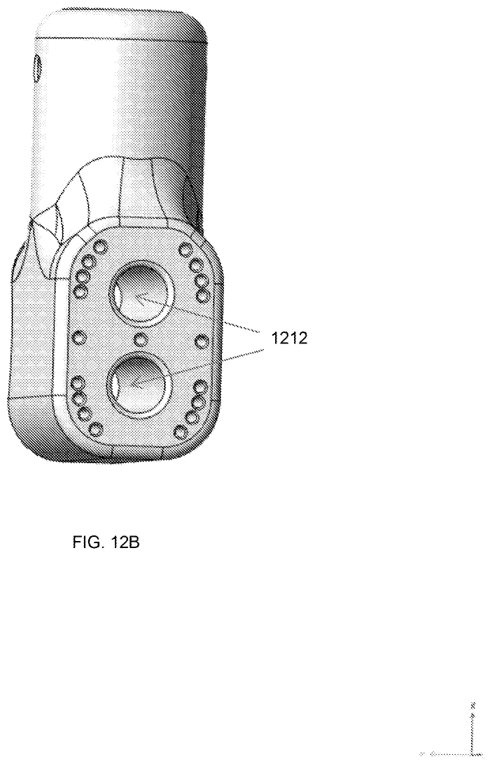

FIGS. 8 to 11 are photographs taken on 9 Jul. 2021 starting at 2:27 PM at the LSUHSC Orthopedic Surgery training lab on the 5th floor of 2020 Gravier Street. Both Dr. Peter Krause and Dr. Charles E. Taylor were present for the testing. FIG. 8 shows a photograph of Initial placement of R2 design on cadaver. FIG. 9 shows a photograph of Initial k-wire anchoring on R2. FIG. 10 shows a photograph of Alternate view of initial k-wire placement on R2. FIG. 11 shows a photograph of K-wires and trocar set. It was found that the device was able to articulate with the tooling, structurally withstand the actions of setting the screws, and can perform under fluoroscopy imaging. We can validate the dimensions and tolerancing of the surgical components.

### Other Embodiments

### Krause_Drillguide_R1A

Design with modification of the handle implementation. The handle utilizes a circular pocket seat with retainment pin, with the handle portion being an acetal Delrin rod (½″ diameter) cut to length. The drill bushings are drawn for 4.25 mm ID×8 mm Tall Steel (Rockwell C61); chosen for a 4.2 mm drill. Material: VeroWhite (non-biocompatible)

### Krause_Drillguide_R1B

Design modified to anchor handle to side of drill guide to handle larger forces that can be imparted by drill shaft interaction with drill guide. The handle utilizes a circular pocket seat with retainment pin, with the handle portion being an acetal Delrin rod (½″ diameter) cut to length. The drill guides are drawn for 4.25 mm ID×8 mm Tall Steel (Rockwell C61); chosen for a 4.2 mm drill. Material: VeroWhite (non-biocompatible)

### Example 3

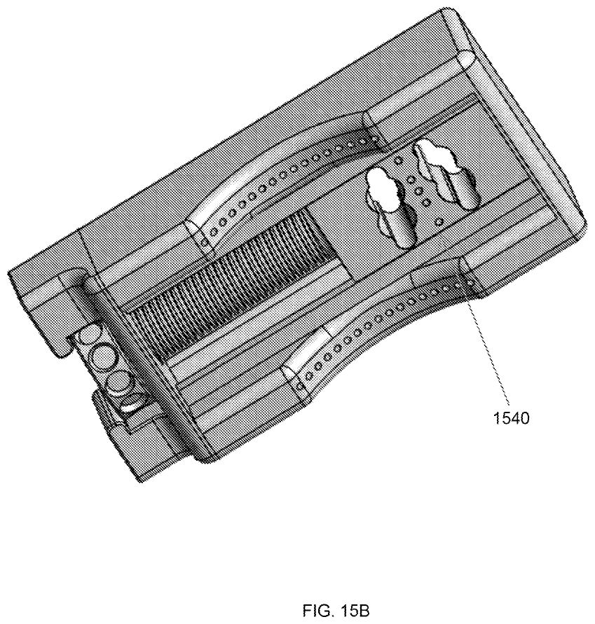

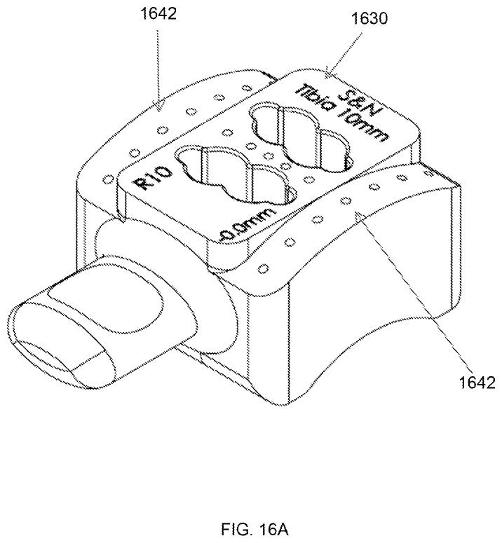

FIG. 15A and FIG. 15B show perspective views of a Krause_DrillGuide_R8 blocking screw alignment device (hereinafter referred to as the “R8 design” or the “R8 device”), under an embodiment. The R8 device features proximal 1510 and distal 1520 alignment (or tool access holes for the alignment and placement of blocking screws. FIG. 15A and FIG. 15B illustrate opposing series of K-wire holes 1530, 1540 laterally extending along opposing sides of the device. FIG. 15A and FIG. 15B also show a series of K-wire holes longitudinally extending between alignment hole pairings. The R8 design incorporates a screw mechanism that allows the adjustment of the alignment holes in the medial-to-lateral direction, providing some adjustment in the OR of the alignment after the frame had been anchored with k-wires.

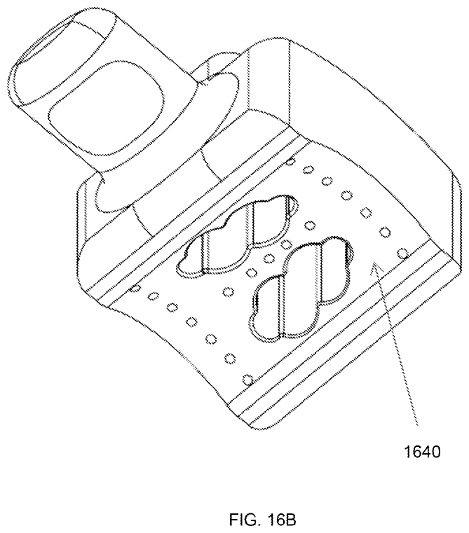

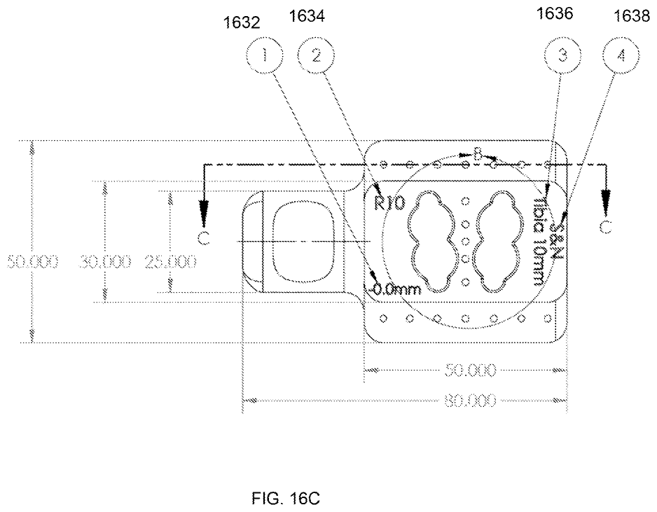

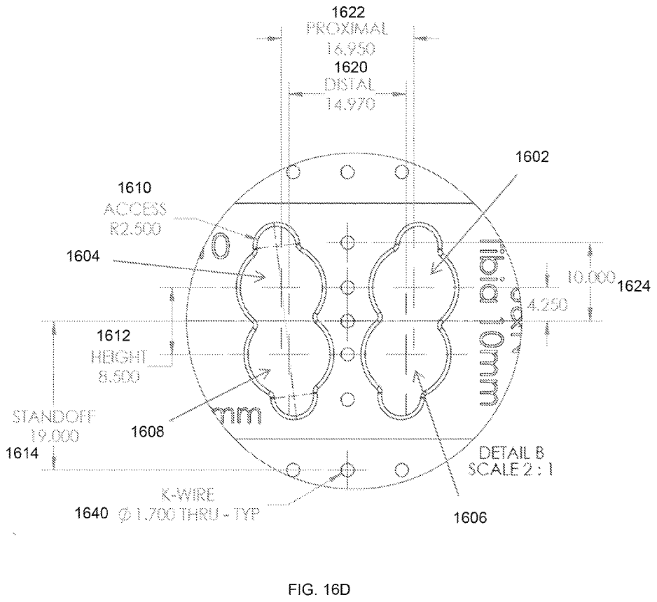

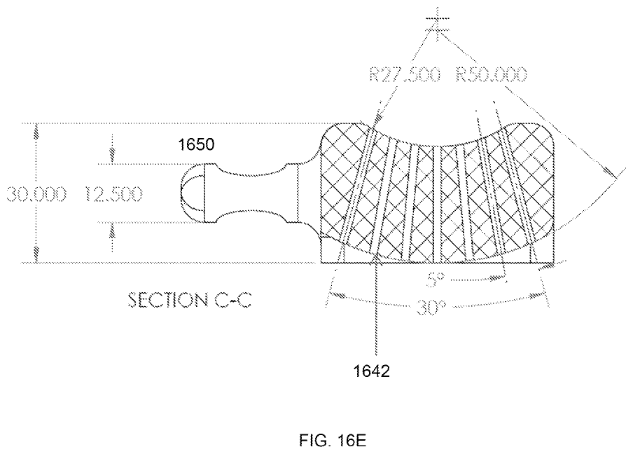

FIG. 16A and FIG. 16B show perspective views of Krause_DrillGuide_R10 blocking screw alignment device (hereinafter referred to as the “R10 design” or the “R10 device), under an embodiment. FIG. 16C shows a top view of the R2 device. FIG. 16D provides a partial top view of the R2 device and features a detailed view of the alignment (or tool access) holes for the blocking screw placement.

As seen in FIG. 16D, the hole pair on top (1602, 1604) are for the Proximal positioning of the nail. For the tibial nail, these would be near the knee. Both pair (1602, 1604, 1606, 1608) are for the Distal screws. The spacing dimensions are based on the use of a 5 mm screw from each vendor and account for both the diameter of the screw and the A-P width of the nail. The Access dimension (1610) controls the extension of the slot to allow for a scalpel to access the skin surface to provide the initial cut for the tissue sleeve to easily penetrate the surface. The Height (1612) spacing of the holes allows for separation of the Distal (1620) and Proximal (1622) holes, while allowing for enough contact surface to grip the tissue sleeves. The distance between Distal hole centers comprises 14.970 mm. The distance between Proximal hole centers comprises 16.950 mm. The Standoff (1614) distance controls the spacing of the K-wire hole array from the tissue sleeve holes to enable the K-Wire driver head to have enough clearance such as not to collide with the tissue sleeves. The K-Wire hole diameter (1640) of 1.7 mm is for a 1.6 mm Stryker K-Wire product. The centered hole k-wire hole array 1624 between the tissue sleeve aligner holes comprises an arbitrary pattern with a center hole and two additional holes at 4.25 & 10 mm respectively.

As shown in FIG. 16C, the R2 device features a raised 30×50 mm rectangularly shaped block 1630. The inscriptions on the block contain a ‘Change from nominal spacing’ dimension 1632 that can be altered to tighten the fit (negative value) of the screws against the nail or loosen the fit (positive value). The Version 1634 is the revision of the design currently embodied. The Nail Type and Size 1636 denote the geometry of the nail (e.g. Tibial 10 mm). The Vendor 1638 is the supplier of the nail product, which can be independent of the screw vendor, but a design may be built with matching screw nail product systems, under an embodiment.

FIG. 16E shows a K-wire hole array 1642 that provides a means of anchoring the device to the patient. The array comprises opposing series of holes, wherein each series is patterned in a radial fashion with the focal point being below the intermedullary canal to prevent wire collisions when using more than 1 hole on a side of the device. Each series features K-wire holes separated from each other at five degrees and from end to end at 30 degrees. The R10 device also features a curved skin surface 1640 that allows better placement of the k-wires and blocking screws.

Under an embodiment, the R10 device may be configured for attachment to a handle. As illustrated in FIG. 16E, an attachment component extends laterally from the R2 device and is configured to couple with a handle. The attachment components is 25 mm in width, 30 mm in length, and 12.5 mm in height with an arcuate recessed portion on its upper and lower surface. The attachment component may attach to a handle using a press fit. Under alternative embodiments, R10 uses a retaining pin, a threaded, coupling, or spring-locked mechanisms. Under an alternative embodiment, the R10 device may not provide any attachment component for attaching to a handle.

Under an embodiment, the R10 device incorporates radio-opaque alignment structures (i.e. sights) that are be used to orient the device under fluoroscopy against the anatomy of the patient).

Under an embodiment, the R10 device incorporates electronics to sense tilt and orientation on the device between blocking screw placements to ascertain whether the device was moved between screw placements; this could lead to the second screw not being within the tolerance of fit the device is seeking to achieve.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A surgical alignment device comprising an alignment head, wherein the alignment head comprises:
at least two tool access holes;
a k-wire hole array comprising holes parallel to the tool access holes; and
a handle attachment pocket perpendicular to the tool access holes.

2. The surgical alignment device of claim 1, wherein the k-wire hole array comprises an arrangement of holes in an arc pattern surrounding the tool access holes.

3. The surgical alignment device of claim 2, wherein the arc pattern comprises radially distributed groupings of holes about the alignment head.

4. The surgical alignment device of claim 3, wherein the radially distributed groupings comprise at least one first grouping of holes.

5. The surgical alignment device of claim 4, wherein the radially distributed groupings comprise at least one second grouping of holes.

6. The surgical alignment device of claim 5, wherein locations of holes in the at least first grouping are laterally transposed from locations of holes in the at least second grouping.

7. The surgical alignment device of claim 5, wherein locations of holes in the at least first grouping are longitudinally transposed from locations of holes in the at least second grouping.

8. The surgical alignment device of claim 1, wherein the k-wire hole array comprises an arrangement of k-wire holes longitudinally aligned to follow a tibial nail axis.

9. The surgical alignment device of claim 1, wherein the handle attachment pocket further comprises retainment pen holes.

10. The surgical alignment device of claim 9, wherein the handle is attachable to the alignment head with the placement of a retainment pen in the retainment pen holes.

11. A surgical alignment device comprising an alignment head, wherein the alignment head comprises:
a plurality of tool access holes;
a k-wire hole array comprising a first plurality of k-wire holes and a second plurality of k-wire holes, wherein the first plurality of k-wire holes extends radially from a first surface of the alignment head to a second surface of the alignment head, wherein the second plurality of k-wire holes are parallel to the plurality of tool access holes.

12. The surgical alignment device of claim 1, wherein the plurality of tool access holes comprises a first and second pair of tool access holes.

13. The surgical alignment device of claim 12, wherein the first pair of tool access holes comprises a first proximal tool access hole and a first distal tool access hole.

14. The surgical alignment device of claim 13, wherein the second pair of tool access holes comprises a second proximal tool access hole and a second distal tool access hole.

15. The surgical alignment device of 14, wherein centers of the first and second proximal tool access holes laterally oppose each other at a first distance.

16. The surgical alignment device of claim 15, wherein centers of the first and second distal tool access holes laterally oppose each other at a second distance, wherein the first distance is greater than the second distance.

17. The surgical alignment device of claim 11, wherein the first pair of tool access holes are joined in fluid communication through a shared open pathway extending along their respective lengths.

18. The surgical alignment device of claim 11, wherein the second pair of tool access holes are joined in fluid communication through a shared open pathway extending along their respective lengths.

19. The surgical alignment device of claim 11, wherein the first and second pair of tool access holes comprise semi-circular access extensions in respective proximal and distal directions.

20. The surgical alignment device of claim 11, wherein the first plurality of k-wire holes are laterally distributed along opposing sides of the alignment head.

21. The surgical alignment device of claim 11, wherein the second plurality of k-wire holes are longitudinally distributed between the first and second pair of tool access holes.

22. The surgical alignment device of claim 11, wherein the first plurality of k-wire holes are radially separated from each other at five degrees.

23. The surgical alignment device of claim 11, wherein the first plurality of k-wire holes are radially separated end to end at thirty degrees.

24. The surgical alignment device of claim 11, wherein the second plurality of k-wire holes occupy a second plane, wherein the first plurality of k-wire holes occupy two respective parallel planes, wherein the first plane is orthogonal to the two respective parallel planes.

25. The surgical alignment device of claim 11, wherein a handle attachment laterally extends from the alignment head, wherein the handle attachment is configured to receive a handle in a press fit coupling.

26. A method comprising,
configuring an alignment device for placement of at least one blocking screw, wherein the alignment head comprises a plurality of tool access holes, a k-wire hole array comprising a first plurality of k-wire holes and a second plurality of k-wire holes, wherein the first plurality of k-wire holes extends radially from a first surface of the alignment head to a second surface of the alignment head, wherein the second plurality of k-wire holes are parallel to the plurality of tool access holes.

27. The surgical alignment device of claim 26, wherein the plurality of tool access holes comprises a first and second pair of tool access holes.

28. The surgical alignment device of claim 27, wherein the first pair of tool access holes comprises a first proximal tool access hole and a first distal tool access hole.

29. The surgical alignment device of claim 28, wherein the second pair of tool access holes comprises a second proximal tool access hole and a second distal tool access hole.

30. The surgical alignment device of 29, wherein centers of the first and second proximal tool access holes laterally oppose each other at a first distance.

31. The surgical alignment device of claim 30, wherein centers of the first and second distal tool access holes laterally oppose each other at a second distance, wherein the first distance is greater than the second distance.

32. The surgical alignment device of claim 26, wherein the first pair of tool access holes are joined in fluid communication through a shared open pathway extending along their respective lengths.

33. The surgical alignment device of claim 26, wherein the second pair of tool access holes are joined in fluid communication through a shared open pathway extending along their respective lengths.

34. The surgical alignment device of claim 26, wherein the first and second pair of tool access holes comprise semi-circular access extensions in respective proximal and distal directions.

35. The surgical alignment device of claim 26, wherein the first plurality of k-wire holes are laterally distributed along opposing sides of the alignment head.

36. The surgical alignment device of claim 26, wherein the second plurality of k-wire holes are longitudinally distributed between the first and second pair of tool access holes.

37. The surgical alignment device of claim 26, wherein the first plurality of k-wire holes are radially separated from each other at five degrees.

38. The surgical alignment device of claim 26, wherein the first plurality of k-wire holes are radially separated end to end at thirty degrees.

39. The surgical alignment device of claim 26, wherein the second plurality of k-wire holes occupy a second plane, wherein the first plurality of k-wire holes occupy two respective parallel planes, wherein the second plane is orthogonal to the two respective parallel planes.

40. The surgical alignment device of claim 26, wherein a handle attachment laterally extends from the alignment head, wherein the handle attachment is configured to receive a handle in a press fit coupling.

41. A method of guiding blocking screws into a bone of a subject comprising:
placing the surgical alignment device of any one of claims 1-40 on a subject to align with a bone to which at least one blocking screw is to be administered;
anchoring the device on the subject by placement of at least one k-wire; and
inserting a tool through the surgical alignment device.

42. The method of claim 41, wherein the blocking screws are administered prior to a reduction of a bone fracture.

43. The method of claim 41, wherein the blocking screws are administered prior to a nail insertion.

44. The method of claim 41, wherein the bone comprises a tibia.

45. The method of claim 41, wherein the bone comprises a femur.
